(19) United States
(12) Patent Application Publication
(10) Pub. No.: US 2007/0032771 A1
(43) Pub. Date: Feb. 8, 2007

(54) Garments containing elastic nonwovens

(75) Inventors: Jean-Claude Abed, Peine (DE); Manfred Wittner, Braunschweig (DE)

Correspondence Address: O'KEEFE, EGAN & PETERMAN, L.L.P., Building C, Suite 200, 1101 Capital of Texas Highway South, Austin, TX, 78746, US

(73) Assignee: Advanced Design Concepts, GmBH

(21) Appl. No.: 11/440,412

(22) Filed: May 24, 2006

Related U.S. Application Data
(60) Provisional application No. 60/684,146, filed on May 24, 2005.

(51) Int. Cl. A61F 13/15

(52) U.S. Cl. 604/385.22

(57) ABSTRACT

An article of clothing made from an elastic nonwoven web, wherein the elastic nonwoven web comprises a plurality of multicomponent strands having first and second polymer components longitudinally coextensive along the length of the strands, said first component comprising an elastomeric polymer, and said second polymer component comprising a polymer less elastic than the first polymer component.

This application claims priority to U.S. provisional application Ser. No. 60/684,146, filed May 24, 2005, incorporated in its entirety herein by reference.

### FIELD OF THE INVENTION

The invention relates to adult incontinence products, feminine hygiene products, semi-disposable nonwoven garments, and sweat-control garments produced from multi-component strands, processes for producing nonwoven webs, and products using the nonwoven webs. The nonwoven webs of the invention can be produced from multi-component strands including at least two components, a first, elastic polymeric component and a second, extensible and/or less elastic polymeric component.

### BACKGROUND OF THE INVENTION

In recent years there has been a dramatic growth in the use of nonwovens, particularly elastomeric nonwovens composites, in disposable hygiene products. For example, elastic nonwoven composites have been incorporated into bandaging materials, garments, diapers, support clothing, and feminine hygiene products. The incorporation of elastomeric components into these products provides improved fit, comfort and leakage control.

However, the inventors have determined that a need exists for adult incontinence products, feminine hygiene products, semi-disposable products and garments containing elastic nonwovens that possess body shaping properties not provided by current elastic nonwovens composites. The present inventors have recognized that a solution to this problem would be highly desirable.

### SUMMARY OF THE INVENTION

The present invention employs elastic nonwoven webs in forming semi-disposable garments and products made from a plurality of strands comprising at least two polymeric components where one component is elastic and another component is extensible and/or less elastic. This elastic nonwoven can compose all or certain portions of the garments and products.

In one broad respect, this invention is a method for producing a garment, comprising forming at least a portion of the garment from an elastic nonwoven web, wherein the nonwoven web comprises a plurality of multicomponent strands having first and second polymer components longitudinally coextensive along the length of the strands, said first component comprising an elastomeric polymer, and said second polymer component comprising a polymer less elastic than the first polymer component.

In another broad respect, this invention is a feminine hygiene product, comprising an elastic nonwoven web in the form of a feminine hygiene product and adapted for receipt of a disposable absorbent device, wherein the elastic nonwoven web comprises a plurality of multicomponent strands having first and second polymer components longitudinally coextensive along the length of the strands, said first component comprising an elastomeric polymer, and said second polymer component comprising a polymer less elastic than the first polymer component.

In another broad respect, this invention is a semi-disposable article of clothing made from an elastic nonwoven web, wherein the elastic nonwoven web comprises a plurality of multicomponent strands having first and second polymer components longitudinally coextensive along the length of the strands, said first component comprising an elastomeric polymer, and said second polymer component comprising a polymer less elastic than the first polymer component.

In another broad respect, this invention is an adult incontinence garment, comprising an elastic nonwoven web in the form of a feminine hygiene product and adapted for receipt of a disposable absorbent device, wherein the elastic nonwoven web comprises a plurality of multicomponent strands having first and second polymer components longitudinally coextensive along the length of the strands, said first component comprising an elastomeric polymer, and said second polymer component comprising a polymer less elastic than the first polymer component.

In one embodiment, optionally, the elastic nonwoven web has been subjected to stretching, such as biaxial stretching, before or after formation of the garment or product. The elastic nonwoven can be optionally stretched with heating. Stretching can serve to decrease the basis weight of the nonwoven web and/or to modify the elastic properties. For example, a tenter frame can be used to stretch the web in the cross machine direction (CD) while simultaneously or sequentially stretching the web in the machine direction (MD) using differential speeds produces a unexpectedly and substantial lowering of the basis weight relative to stretching by other methods. It should be noted that cross machine direction generally refers to the width of a fabric in a direction generally perpendicular to the direction in which it is produced, as opposed to machine direction which refers to the length of a fabric in the direction in which it is produced. For example, using this biaxial stretching, a 200% biaxial stretch at room temperature led to a 30% decrease in basis weight in contrast to a 400% stretch using ring rollers (incremental stretching) at room temperature led to only a 10% decrease in basis weight. Even if the basis weight is not reduced significantly (e.g., less than or equal to 10% reduction), it has been found additionally that the use of biaxial stretching, under the conditions set forth, can improve elastic properties (increased extensional force, decreased set, decreased stress relaxation, and increased retractive force).

The present invention is generally directed to elastic nonwoven garments and products, and methods for producing elastic nonwoven such garments and products. The elastic nonwovens can be made by a variety of processes, such as by melt spinning a plurality of multicomponent strands having first and second polymer components longitudinally coextensive along the length of the filament. The first component is formed from an elastomeric polymer and the second component is formed from an extensible and/or less elastomeric polymer. The melt spun strands are formed into a nonwoven web which is subsequently bonded. The bonded web may be optionally stretched to reduce the basis weight and denier of the nonwoven without diminishing the elastic and physical properties of the nonwoven materials beyond acceptable ranges, such as by post mechanically stretching a pre-made thermopoint bonded elastic nonwoven in the machine direction, transverse direction, or preferably both directions. The nonwoven can be preheated prior to or during the stretching, or not heated.

With respect to the multicomponent strands, the first and second components can be derived from any of a wide variety of polymers. In one embodiment of the invention, the first polymer component is formed from an elastomeric polyurethane, elastomeric styrene block copolymer, polyester, or an elastomeric polyolefin such as Vistamaxx from Exxon and the second polymer component is formed from a polyolefin that is less elastic than the first component.

In one broad respect, this invention is a semi-disposable garment, which comprises: an adult incontinence product which comprises a pant-like garment adapted to wrap around a torso of a wearer to hold the garment in place, including a crotch portion that extends from the waist between the legs of the wearer, wherein the crotch portion is made of elastic nonwoven fibers, such as bonded elastic nonwoven webs formed from the multicomponent strands having first and second polymer components longitudinally coextensive along the length of the filament.

In one embodiment, the elastic nonwoven fabric used in the practice of this invention can be made by stretching an elastic nonwoven web in at least one direction, preferably by biaxially stretching the web, at an elevated temperature to reduce the basis weight of the web, wherein the nonwoven web comprises a plurality of multicomponent strands having first and second polymer components longitudinally coextensive along the length of the strands, said first component comprising an elastomeric polymer, and said second polymer component comprising a polymer less elastic than the first polymer component.

In one embodiment, the elastic nonwoven web can be formed by melt spinning a plurality of multicomponent strands having first and second polymer components longitudinally coextensive along the length of the strands, said first component comprising an elastomeric polymer, and said second polymer component comprising a non-elastomeric polymer; forming the multicomponent strands into a nonwoven web; and multipoint bonding the strands to form a coherent bonded nonwoven web; and optionally stretching the bonded nonwoven in at least one direction.

In another broad respect, this invention is an adult incontinence garment, a feminine hygiene garment, or a semi-disposable athletic garment which comprises at least a portion of such garment formed of the elastic nonwoven fabric or which comprises a plurality of layers, wherein at least one of said layers comprises the nonwoven fabric described above.

The fibers, articles, or garments of the present invention have utility in a variety of additional applications. Suitable applications include, for example, but are not limited to, disposable personal hygiene products (e.g. training pants, diapers, absorbent underpants); disposable garments (e.g. industrial apparel, coveralls, head coverings, underpants, pants, shirts, gloves, socks and the like); infection control/clean room products (e.g. surgical gowns and drapes, face masks, head coverings, surgical caps and hood, shoe coverings, boot slippers, wound dressings, bandages, sterilization wraps, wipers, lab coats, coverall, pants, aprons, jackets), and durable and semi-durable applications such as bedding items and sheets, furniture dust covers, apparel interliners, car covers, and sports or general wear apparel.

The garments, products, and articles of this invention advantageously possess one or more of the following properties: the look and feel of knitted fabric, functional elasticity (2-dimensional), high stretch, breathability, and washability (e.g., at least 5 wash/dry cycles). Thus, for example, adult incontinence garments of this invention could provide functional absorptivity in the form, look, and functionality of a standard knitted textile garment.

### BRIEF DESCRIPTION OF THE DRAWINGS

FIGS. 1 and 2 illustrate a representative adult incontinence garment of this invention.

FIG. 3-8 illustrate representative feminine hygiene products of this invention.

### DETAILED DESCRIPTION OF THE INVENTION

Nonwovens are commonly made by melt spinning thermoplastic materials. Such nonwovens are called “spunbond” or “meltblown” materials and methods for making these polymeric materials are also well known in the field. Spunbonded materials are preferred in this invention due to advantageous economics. While spunbond materials with desirable combinations of physical properties, especially combinations of softness, strength and durability, have been produced, significant problems have been encountered. The nonwovens employed in this invention are typically conjugate fibers and typically bicomponent fibers. In one embodiment the nonwoven is made from bicomponent fibers having a sheath/core structure. Representative bicomponent, elastic nonwovens and the process for making them, suitable for this invention, are given by Austin in WO 00/08243, incorporated herein by reference in its entirety.

As used herein, the term “strand” is being used as a term generic to both “fiber” and filament”. In this regard, “filaments” are referring to continuous strands of material while “fibers” mean cut or discontinuous strands having a definite length. Thus, while the following discussion may use “strand” or “fiber” or “filament”, the discussion can be equally applied to all three terms.

Specifically, what is about to be described hereinbelow for the elastic nonwoven are what we would define as “chemically” elastic fibers. To those skilled in the art it will be readily apparent the distinction of these fibers from the less elastic, 1-dimensionally elastic, “physical” or “mechanical” elastic nonwovens produced via heat stretching of an otherwise essentially inelastic nonwoven.

Briefly, the bicomponent strands used to make the elastic nonwoven are typically composed of a first component and a second component. The first component is an “elastic” polymer(s) which refers to a polymer that, when subjected to an extension, deforms or stretches within its elastic limit (i.e., it retracts when released). Many fiber forming thermoplastic elastomers are known in the art and include polyurethanes, block copolyesters, block copolyamides, styrenic block polymers, and polyolefin elastomers including polyolefin copolymers. Representative examples of commercially available elastomers for the first (inner) component include the KRATON polymers sold formerly by Kraton Corp.; ENGAGE elastomers (sold by Dupont Dow Elastomers), VISTAMAXX (produced by Exxon-Mobile Corp.) polyolefin elastomers; and the VECTOR polymers sold by DEXCO. Other elastomeric thermoplastic polymers include polyurethane elastomeric materials (“TPU”), such as PELLETHANE sold by Dow Chemical, ELASTOLLAN sold by BASF, ESTANE sold by B.F. Goodrich Company; polyester elastomers such as HYTREL sold by E.I. Du Pont De Nemours Company; polyetherester elastomeric materials, such as ARNITEL sold by Akzo Plastics; and polyetheramide materials, such as PEBAX sold by Elf Atochem Company. Heterophasic block copolymers, such as those sold by Montel under the trade name CATALLOY are also advantageously employed in the invention. Also suitable for the invention are polypropylene polymers and copolymers described in U.S. Pat. No. 5,594,080, incorporated herein by reference.

The second component is also a polymer(s), preferably a polymer which is extensible. Any thermoplastic, fiber forming, polymer would be possible as the second component, depending on the application. Cost, stiffness, melt strength, spin rate, stability, etc will all be a consideration. The second component may be formed from any polymer or polymer composition exhibiting inferior elastic properties in comparison to the polymer or polymer composition used to form the first component. Exemplary non-elastomeric, fiber-forming thermoplastic polymers include polyolefins, e.g. polyethylene (including LLDPE), polypropylene, and polybutene, polyester, polyamide, polystyrene, and blends thereof. The second component polymer may have elastic recovery and may stretch within its elastic limit as the bicomponent strand is stretched. However, this second component is selected to provide poorer elastic recovery than the first component polymer. The second component may also be a polymer which can be stretched beyond its elastic limit and permanently elongated by the application of tensile stress. For example, when an elongated bicomponent filament having the second component at the surface thereof contracts, the second component will typically assume a compacted form, providing the surface of the filament with a rough appearance.

In order to have the best elastic properties, it is advantageous to have the elastic first component occupy the largest part of the filament cross section. In one embodiment, when the strands are employed in a bonded web environment, the bonded web has a root mean square average recoverable elongation of at least about 65% based on machine direction and cross direction recoverable elongation values after 50% elongation and one pull. The root mean square average recoverable elongation is the square root of the sum of (percent recovery in the machine direction)²+percent recovery in the cross machine direction)².

The second component is typically present in an amount less than about 50 percent by weight of the strand, with between about 1 and about 20 percent in one embodiment and about 5-10 percent in another embodiment, depending on the exact polymer(s) employed as the second component.

In one respect, where the second component is substantially not elastic resulting in the strand being not elastic as a whole, in one embodiment the second component is present in an amount such that the strand becomes elastic upon stretching of the strand by an amount sufficient to irreversibly alter the length of the second component.

Suitable materials for use as the first and second components are selected based on the desired function for the strand. Preferably, the polymers used in the components of the invention have melt flows from about 5 to about 1000. Generally, the meltblowing process will employ polymers of a higher melt flow than the spunbonded process.

These bicomponent strands can be made with or without the use of processing additives. In the practice of this invention, blends of two or more polymers can be used for either the first component or second component or both.

The first (the elastic component of the present invention) and second components may be present within the multicomponent strands in any suitable amounts, depending on the specific shape of the fiber and end use properties desired. In advantageous embodiments, the first component forms the majority of the fiber, i.e., greater than about 50 percent by weight, based on the weight of the strand (“bos”). For example, the first component may beneficially be present in the multicomponent strand in an amount ranging from about 80 to 99 weight percent bos, such as in an amount ranging from about 85 to 95 weight percent bos. In such advantageous embodiments, the non-elastomeric component would be present in an amount less than about 50 weight percent bos, such as in an amount of between about 1 and about 20 weight percent bos. In beneficial aspects of such advantageous embodiments, the second component may be present in an amount ranging from about 5 to 15 weight percent bos, depending on the exact polymer(s) employed as the second component. In one advantageous embodiment, a sheath/core configuration having a core to sheath weight ratio of greater than or equal to about 85:15 is provided, such as a ratio of 95:5.

The shape of the fiber can vary widely. For example, typical fiber has a circular cross-sectional shape, but sometimes fibers have different shapes, such as a trilobal shape, or a flat (i.e., “ribbon” like) shape. Also the fibers, even though of circular cross-section, may assume a non-cylindrical, 3-dimentional shape, especially when stretched and released (self-bulking or self-crimping to form helical or spring-like fibers).

For the inventive elastic fibers disclosed herein, the diameter can be widely varied. The fiber denier can be adjusted to suit the capabilities of the finished article. Expected fiber diameter values would be: from about 5 to about 20 microns/filament for melt blown; from about 10 to about 50 micron/filament for spunbond; and from about 20 to about 200 micron/filament for continuous wound filament.

Basis weight refers to the area density of a non-woven fabric, usually in terms of g/m² or oz/yd². Acceptable basis weight for a nonwoven fabric is determined by application in a product. Generally, one chooses the lowest basis weight (lowest cost) that meets the properties dictated by a given product. For elastomeric nonwovens one issue is retractive force at some elongation, or how much force the fabric can apply after relaxation at a certain extension. Another issue defining basis weight is coverage, where it is usually desirable to have a relatively opaque fabric, or if translucent, the apparent holes in the fabric should be of small size and homogeneous distribution. The most useful basis weights in the nonwovens industry for disposable products range from ½ to 4.5 oz/yd² (17 to 150 g/m², or gsm). Some applications, such as durable or semi-durable products, may be able to tolerate even higher basis weights. It should be understood that low basis weight materials may be adventitiously produced in a multiple beam construction. That is, it may be useful to produce an SMS (spunbond/meltblown/spunbond) composite fabric where each of the individual layers have basis weights even less than 17 gsm, but it is expected that the preferred final basis weight will be at least 17 gsm.

A nonwoven composition or article is typically a web or fabric having a structure of individual fibers or threads which are randomly interlaid, but not in an identifiable manner as it is the case for a woven or knitted fabric.

The first and second polymeric components can optionally include, without limitation, pigments, dyes, hydrophilicity modifiers, antioxidants, stabilizers, surfactants, waxes, flow promoters, solid solvents, particulates and material added to enhance processability of the composition.

It should be appreciated that an elastic material or elastic-like nonwoven, as applicable to this invention, typically refers to any material having a root mean square average recoverable elongation of about 65% or more based on machine direction and cross-direction recoverable elongation values after 50% elongation of the web and one pull. The extent that a material does not return to its original dimensions after being stretched and immediately released is its percent permanent set. According to ASTM testing methods, set and recovery will add to 100%. Set is defined as the residual relaxed length after an extension divided by the length of extension (elongation). For example, a one inch gauge (length) sample, pulled to 200% elongation (two additional inches of extension from the original one inch gauge) and released might a) not retract at all so that the sample is now three inches long and will have 100% set ((3″_{end}−1″ᵢₙᵢₜᵢₐₗ)/2″ₑₓₜₑₙₛᵢₒₙ), or b) retract completely to the original one inch gauge and will have 0% set ((1″_{end}−1″ᵢₙᵢₜᵢₐₗ)/2″ₑₓₜₑₙₜᵢₒₙ), or c) will do something in between. An often used and practical method of measuring set is to observe the residual strain (recovery) on a sample when the restoring force or load reaches zero after it is released from an extension. This method and the above method will only produce the same result when a sample is extended 100%. For example, as in the case above, if the sample did not retract at all after 200% elongation, the residual strain at zero load upon release would be 200%. Clearly in this case set and recovery will not add to 100%.

By contrast, a non-elastic nonwoven does not meet these criteria. Specifically, a non-elastic nonwoven would be expected to demonstrate less than 50%, more likely less than 25%, recovery when extended to 50% of its original length. Moreover, non-elastic nonwovens are typically described by a tensile curve that shows extensive yielding prior to break. In this regard the nonwoven will show a rapid increase in stress at small extensions followed by a near maximum, approximately constant stress at the yield point and during continued extension until the nonwoven ruptures. Prior to rupture a release of the sample results in an extensively elongated, non-retracted nonwoven.

Nonwoven webs can be produced from the multicomponent strands of the invention by any technique known in the art. A class of processes, known as spunbonding is one common method for forming nonwoven webs. Examples of the various types of spunbonded processes are described in U.S. Pat. No. 3,338,992 to Kinney, U.S. Pat. No. 3,692,613 to Dorschner, U.S. Pat. No. 3,802,817 to Matsuki, U.S. Pat. No. 4,405,297 to Appel, U.S. Pat. No. 4,812,112 to Balk, and U.S. Pat. No. 5,665,300 to Brignola et al. In general, traditional spunbonded processes include:

a) extruding the strands from a spinneret;

b) quenching the strands with a flow of air which is generally cooled in order to hasten the solidification of the molten strands;

c) attenuating the filaments by advancing them through the quench zone with a draw tension that can be applied by either pneumatically entraining the filaments in an air stream or by wrapping them around mechanical draw rolls of the type commonly used in the textile fibers industry;

d) collecting the drawn strands into a web on a foraminous surface; and

e) bonding the web of loose strands into a fabric.

This bonding can use any thermal, chemical or mechanical bonding treatment known in the art to impart coherent web structures. Thermal point bonding may advantageously be employed in the practice of this invention. Various thermal point bonding techniques are known, with the most preferred utilizing calender rolls with a point bonding pattern. Any pattern known in the art may be used with typical embodiments employing continuous or discontinuous patterns. Preferably, the bonds cover between 6 and 30 percent, and most preferably, 16 percent of the layer is covered. By bonding the web in accordance with these percentage ranges, the filaments are allowed to elongate throughout the full extent of any optional stretching while the strength and integrity of the fabric can be maintained. In alternative aspects of the invention, bonding processes that entangle or intertwine the strands within the web may be employed. An exemplary bonding process which relies upon entanglement or intertwining is hydroentanglement.

All of the spunbonded processes of this type can be used to make the elastic fabric of this invention if they are outfitted with a spinneret and extrusion system capable of producing multicomponent strands. However, one preferred method involves providing improved web laydown via a vacuum located under the forming surface. This method provides for a continually increasing strand velocity to the forming surface, and so provides little opportunity for the elastic strands to snap back.

Another class of process, known as meltblowing, can also be used to produce the nonwoven fabrics of this invention. This approach to web formation is described in NRL Report 4364 “Manufacture of Superfine Organic Fibers” by V. A. Wendt, E. L. Boone, and C. D. Fluharty and in U.S. Pat. No. 3,849,241 to Buntin et al. Conventional meltblowing process generally involve:

a.) Extruding the strands from a spinneret.

b.) Simultaneously quenching and attenuating the polymer stream immediately below the spinneret using streams of high velocity heated air. Generally, the strands are drawn to very small diameters by this means. However, by reducing the air volume and velocity, it is possible to produce strand with deniers similar to common textile fibers.

c.) Collecting the drawn strands into a web on a foraminous surface. Meltblown webs can be bonded by a variety of means, but often the entanglement of the filaments in the web or the autogeneous bonding in the case of elastomers provides sufficient tensile strength so that it can be wound onto a roll. Thermopoint bonding is advantageously used in the practice of this invention.

Any meltblowing process which provides for the extrusion of multicomponent strands such as that set forth in U.S. Pat. No. 5,290,626 can be used to practice this invention.

The fabric of the invention may also be treated with other treatments such as antistatic agents, alcohol repellents and the like, by techniques that would be recognized by those skilled in the art.

After bonding the nonwoven web, the material is optionally stretched, such as by using biaxial stretching. The biaxially stretched can be conducted under elevated temperature. The biaxial stretching can serve to affect the basis weight reduction. Typically the stretching is accomplished by use of tenter frame stretching in the cross direction in combination with or subsequent to differential speed stretching in the machine direction. For example, a thermopoint bonded elastic nonwoven web is fed by a suitable conveyor to fabric stretching means in the form of a conventional tenter apparatus or frame. At a first position, two endless chains respectively engage the edge portions of the web with a series of hooks or clamps mounted and simultaneously convey the thus engaged fabric to a second position and stretch the fabric web transversely relative to its direction of travel. During the stretching the web may also heated to a temperature of about 20 C (room temperature), in one embodiment to about 40 C, and in another embodiment to about 60 C. Optimal heating temperature selection is a complicated function of, amongst others, the speed of the fabric, the construction of the fibers, the materials used, and the final properties (basis weight and elastomeric) desired. Generally the temperature will be less than or about equal to a temperature that could be used to thermopoint bond the web. Any available form of tenter frame may be used in the practice of the present invention. The tenter frame selected should, however, be one which provides even air flow across the web. The tenter frame should also be equipped with overfeed means to allow as much as 30% overfeed, so that the fabric can be relaxed during processing to permit controlled shrinkage. Tenter frames may be composed of successive chambers or zones, provided with separate means for circulating hot air therethrough and it may be desirable in certain circumstances involving the practice of the invention to vary the temperature of the circulating air. In general, the web is stretched at least 50% during this step. In one embodiment, the web is stretched using the tenter frame at least 100%. Subsequently or simultaneously to transverse stretching, the web is typically stretched using differential speeds of the rollers in the machine direction. In this regard, “biaxial” stretching refers to stretching ultimately in both the CD and MD. For example, where there is a 2× difference in speed between the feed and take up rollers, a 100% stretch of the web occurs in the machine direction. Other stretch percentages may be employed in the practice of this invention. It should be appreciated that the web may also be subjected to heating during the machine direction stretch, at temperatures generally the same as the temperature during cross direction stretching. It should be appreciated that the stretching can occur in a single step, or can be performed by multiple stretches to affect the desired stretch and basis weight. For example, the nonwoven can be subjected to a 100% stretch followed by a 50% stretch, instead of a single 200% stretch (to achieve a 3× overall stretch). If stretched, the basis weight of the nonwoven web is typically reduced at least 10%. In one embodiment, the basis weight is reduced at least 20%. In another embodiment, the basis weight is reduced about 30% or even higher.

The nonwoven fibers and articles of this invention can optionally be colored, as by dyeing or inclusion of a pigment. Conventional methods can be employed to dye the nonwovens, articles, and fibers of this invention. Alternatively, the nonwovens, articles, and fibers can be treated with pigments for coloration. For instance, the nonwovens, articles, and fibers may be dyed in a dye both using conventional dyes and disperse dye techniques. In some cases, additives may be added to the nonwovens, articles, and fibers (hereafter generally referred to as fibers for purposes of coloration) to enhance the dyeability. Generally, the dye is applied in the form of a dye solution so that it can be readily applied by dipping the strand/fiber through a trough, for example, containing the dye solution, or by spraying the dye solution on the fiber, or by using a cascading roll technique. As is common, the dye solution can be in the form of a print paste, from which the dyeing is typically conducted by roller printing or screen printing. The fibers can be dyed multiple times using one or more dyeing techniques.

Aqueous dye baths typically have a pH of from about 2 to about 11, generally between about 2 to about 6 for acid dyes. The pH may be adjusted if desired using a variety of compounds, such as formic acid, acetic acid, sulfamic acid, citric acid, phosphoric acid, nitric acid, sulfuric acid, monosodium phosphate, tetrasodium phosphate, trisodium phosphate, ammonium hydroxide, sodium hydroxide, and combinations thereof. Use of a surfactant can be used to aid in dispersing sparingly water soluble disperse dyes in the dye baths. Typically, nonionic surfactants can be employed for this purpose. During the dying step, the dye bath may be agitated to hasten the dyeing ratio. The dyeing step can be carried out at a variety of temperatures, with higher temperatures generally promoting the rate of dyeing.

Another technique known in the art is jet dyeing, which permits high-temperature dyeing and impingement of the dye onto the moving fabric through use of a venturi jet system. Carriers permit faster dyeing at atmospheric pressure and below 100° C. The carriers are typically organic compounds that can be emulsified in water and that affinity for the fiber. Representative examples of such carriers include aromatic hydrocarbons such as diphenyl and methylnaphthalene, phenols such as phenylphenol, chlorinated hydrocarbons such as dichloro- and tricolor-benzene, and aromatic esters such as methyl salicylate, butyl benzoate, diethylphthalate, and benzaldehyde. Carriers are generally removed after dyeing.

Subsequent to dyeing, using a dye mixture with additives above, dry heat may be applied to the fibers at a wide range of elevated temperatures to cause the dye to penetrate into, and become fixed in, the fiber. The dye fixation step involves exposing the fiber to dry heat, such as in an oven. The temperature can vary up to the melt or glass transition temperature of the composition fiber. Generally, higher drying temperatures result in shorter drying times. Typically, the heating time is from about 1 minute to about 10 minutes. Residual dye may then be removed from the fibers.

A disperse dye mixture may thus be applied to the fibers in various ways. The dye mixture may be applied intermittently along the length of yarn formed from fibers using various well known techniques to create a desired effect. A conventional flat screen printing machine may be used for applying the dye mixture to the nonwoven, fabric, or article.

Continuous dyeing is carried out on a dyeing range where nonwoven is continuously passed through a dye solution of sufficient length to achieve initial dye penetration. Some disperse dyes may be sublimated under heat and partial vacuum into polymer fiber by methods known in the art. Printing of nonwovens made in accordance with this invention can be accomplished with disperse dyes by heat transfer printing under pressure with sufficient heating to cause diffusion or disperse dyes into the polyolefin. Block, flat screen, and heat transfer batch processes, and engraved roller and rotary screen printing continuous processes may be used. Different dye solutions may be jet-sprayed in programmed sequence onto nonwovens of this invention as the fabric passes under the jets to form patterns. Dye solution may be metered and broken or cut into a pattern of drops that are allowed to drop on a dyed nonwoven passing underneath to give a diffuse over-dyed pattern. Different styling effects can be produced by controlling shade depth on each type of fiber present. Acid, disperse and premetallized dyes, or combinations thereof, depending upon the fibers present, can be employed to obtain styling effects.

There are many commercially available disperse dyes. Dyes are classified based on method of application and, to a lesser extent, on chemical constitution by the Society of Dyers and Colorists. Various disperse dyes may be found in the listing “Dyes and Pigments by Color Index and Generic Names” set forth in Textile Chemist and Colorist, July 1992, Vol. 24, No. 7, a publication of the American Association of Textile Chemists and Colorists.

Dyes are intensely colored substances used for the coloration of various substrates, such as paper, plastics, or textile materials. It is believed that dyes are retained in these substrates by physical absorption, by salt or metal-complex formation, or by the formation of covalent chemical bonds. The methods used for the application of dyes to the substrate differ widely, depending upon the substrate and class of dye. It is by applications methods, rather than by chemical constitutions, that dyes are differentiated from pigments. During the application process, dyes lose their crystal structures by dissolution or vaporization. The crystal structures may in some cases be regained during a later stage of the dyeing process. Pigments, on the other hand, retain their crystal or particulate form throughout the entire application procedure.

A large number of dyes, with widely differing properties, are therefore necessary because of the great variety of materials to be dyed. On a worldwide basis, it is believed that several thousand different dyes have achieved commercial significance. Generally, dyes have been classified into groups two ways. One method of classification is by chemical constitution in which the dyes are grouped according to the chromophore or color giving unit of the molecule. A second method of classification is based on the application class of end-use of the dye. The dual classification system used in the color index (CI) is accepted internationally throughout the dye-manufacturing and dye-using industries. In this system, dyes are grouped according to chemical class with a CI number for each chemical compound and according to usage or application class with a CI name for each dye.

A number of spin finishes can be applied to the fibers prior to drawing. Such finishes can be water-based. The spin finishes can be anionic or nonionic, as is well known in the art. Also, the fibers can be finished prior to dyeing, as by texturizing through mechanical crimping or forming, as is well known in the art.

The above-described elastic nonwoven fabric is used to manufacture at least a portion of a garment of this invention. In one embodiment the entire garment is made of one or more of the elastic nonwoven fabrics described herein.

### Adult Incontinence Garments

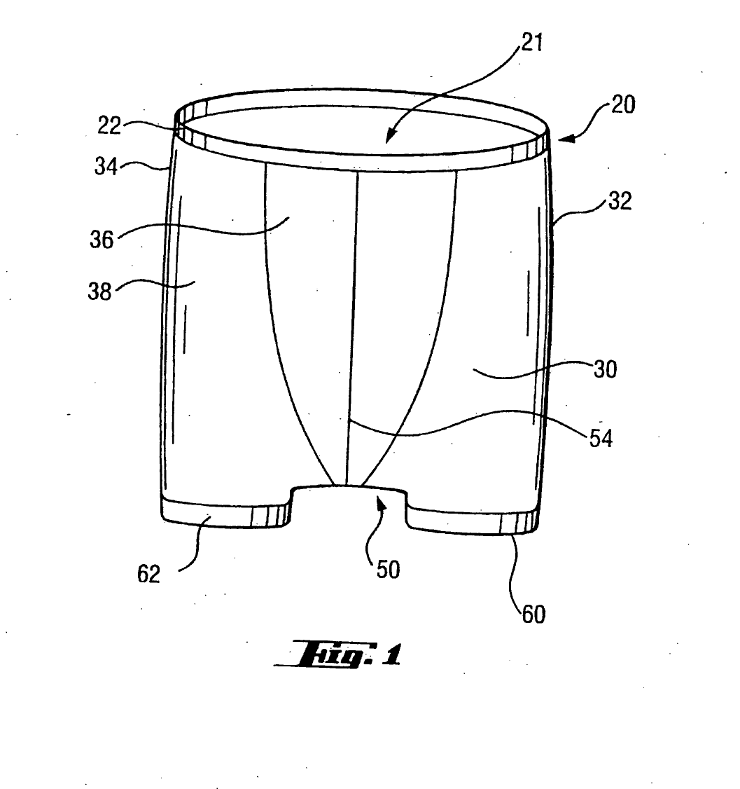

An adult incontinence product generally refers to pant-like garments that are intended to contain body exudates released by the wearer. A representative adult incontinence garment is shown in FIG. 1. They typically consist of a waist portion that wraps around a wearer's torso to hold the garment in place, and a crotch portion that extends from the waist between the wearer's legs. Such products are conventionally made with elastics or other fitting devices in the waist portion to provide fit to thereby hold the garment onto the wearer and to prevent leakage. For example, elastics that are placed in the waists often comprise a number of thin elastic band/strands or films. These bands and films are typically adhesively bonded to one or more nonwovens to impart a more pleasing touch. However, these elastics do not provide body-shaping benefits and do not look and feel like knitted textiles. Furthermore, these products can have a one-piece construction, in which case a garment may be manufactured from a single assembly of parts joined to itself or a multi-piece construction in which the garment is made from several pieces. These garments are typically put on by pulling the product up a person's legs, similar to underwear of pants, but can also be equipped with open seams that are releasable/closable. As used herein, the term “incontinence device” refers to an adsorbent article generally worn by incontinent persons about the lower torso for absorbing and containing bodily fluids, such as urine. As used herein the term “disposable” refers to garments and products that are not intended to be laundered or otherwise restored or reused after use; that is, they are intended to be discarded after a single use. As used herein “semi-disposable” refers to garments and products that are intended to be laundered or otherwise restored or reused at least once, and in one embodiment are capable of being laundered at least 3 without significant loss of properties, for example less than 15% loss of physical properties, preferably capable of being laundered at least 5 times.

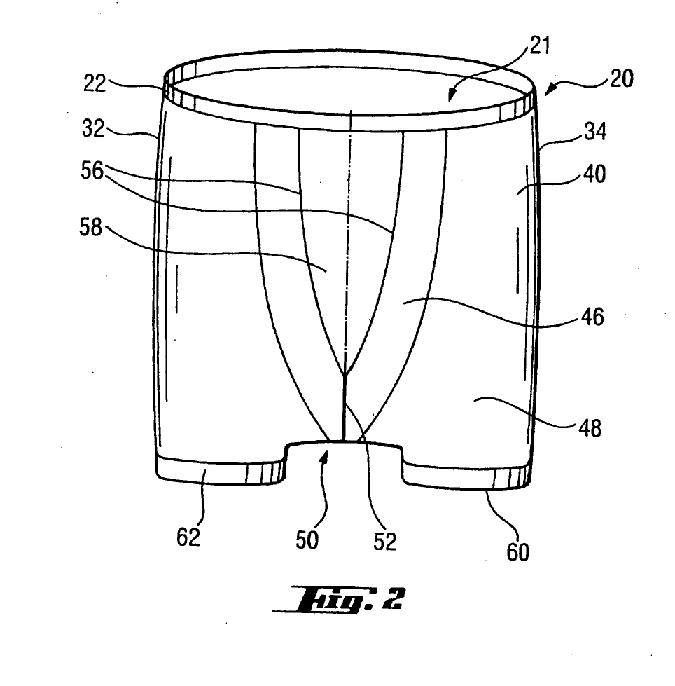

FIGS. 1 and 2 show a representative adult continence garment of this invention. The representative garment 20, which may be referred to as an undergarment, that is depicted in FIGS. 1 and 2 may be used with a wide variety of absorbent articles, such as used in conjunction with an absorbent incontinence device or pad (not shown). The garment depicted in FIGS. 1 and 2 comprises a front panel 30 comprising first section 36 and second section 38, a rear panel 40 comprising first section 46 and second section 48, a crotch region 50, opposed sides 32 and 34, an optional pair of elasticized leg openings 60, optional elasticized leg band 62, and optional pocket 58 that can be used to contain bowel movement, and an optional elasticized waistband 22. The garment 20 may also include a waist opening 21 that allows entry into the garment by the user. The garment 20 may also include longitudinal stretch control member 52 disposed along the longitudinal centerline in the crotch region 50, a front stretch control member 54 disposed in the front panel 30 and extending from the longitudinal stretch control member 52 to the waistband 22, and a rear stretch control member 56 disposed in the rear panel 40 and extending from the longitudinal stretch control member 52 to the waistband 22. At least a portion of the garment 20 is made of the elastic nonwoven of this invention, and in one embodiment the entire garment 20 except for the elasticized leg openings 60 and waistband 22, which are made of the elastic nonwoven of this invention.

The garment 20 can be made by conventional methods well know to those of skill in the art. For example, U.S. Pat. No. 6,287,169, which is hereby incorporated by reference, shows various representative garment configurations that can be employed in the practice of this invention, as well as general methodologies to make such garments. Thus, the garment can be cut, for example, into a single piece configured to be folded and bonded to form the leg openings and torso opening. Alternatively, the garment is made, for example, by forming a tubular form with the crotch area bonded to form the leg openings.

### Feminine Hygiene Garments

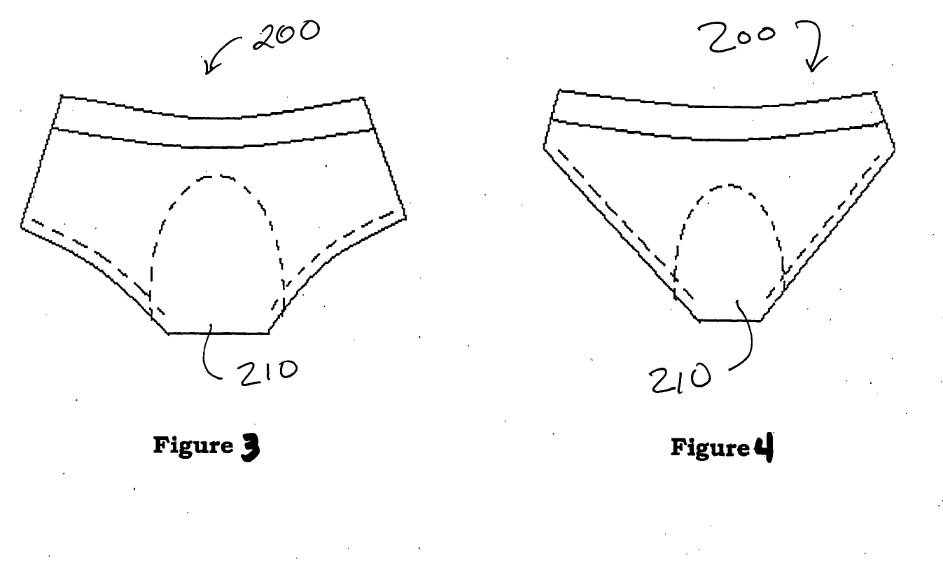

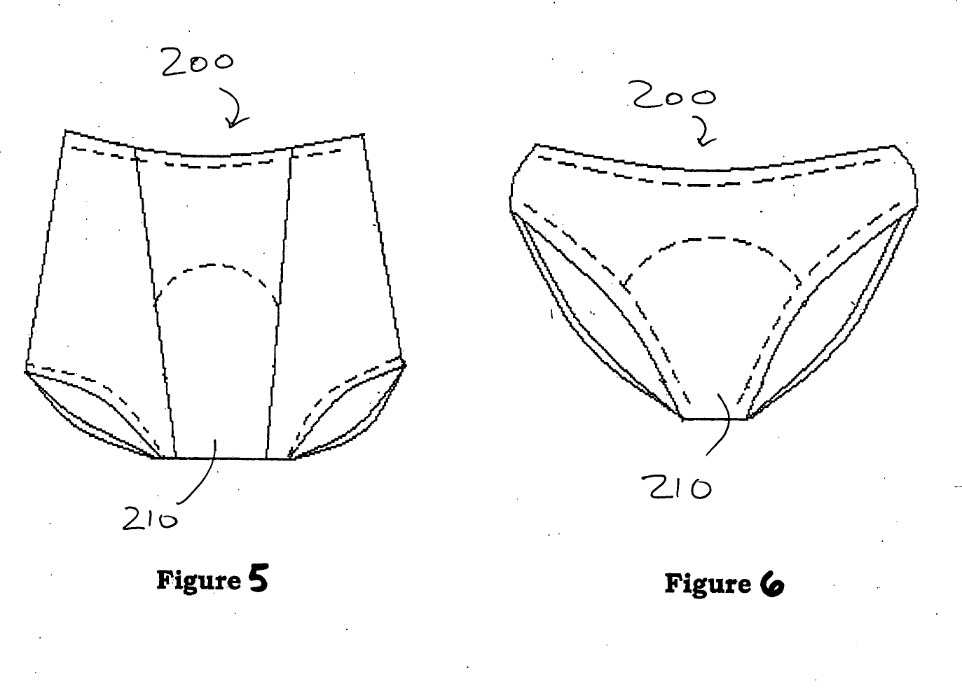

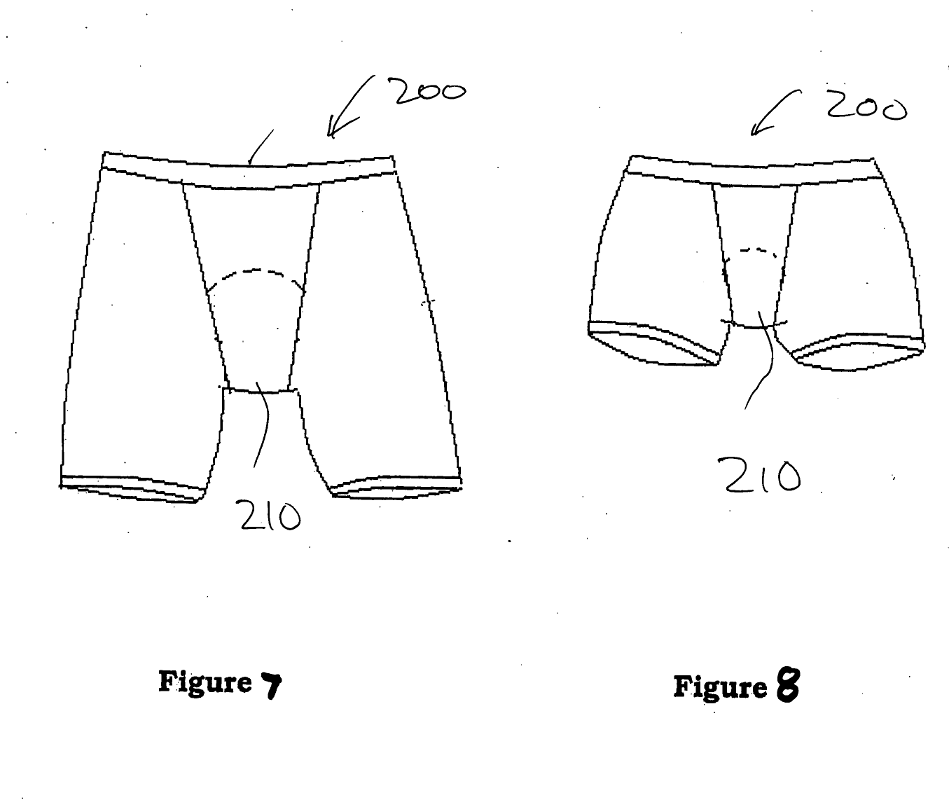

Similar to adult incontinence garments, feminine hygiene garments have been designed to maximize fluid absorption while minimizing obtrusiveness. However, to date there are no products that combine the look and feel of normal cotton underwear with adsorptive capabilities. This invention includes a feminine hygiene garment made at least in part of the elastic nonwoven of this invention. In the feminine hygiene product of this invention, a disposable panty or brief is formed with at least a portion of the panty or brief undergarment being made of elastic nonwoven web. In one embodiment made entirely of the elastic nonwoven. Thus the disposable feminine hygiene undergarments of this invention may resemble the above described adult incontinence garment in overall appearance. Likewise, the feminine hygiene garment may resemble on of the semi-disposable athletic shorts described herein. The feminine hygiene garment is, however, adapted for use with an adsorbent strip or pad as is used in feminine hygiene applications, as one of skill in the art readily understands. Representative feminine hygiene garments 200 are depicted in FIGS. 3-8. Area 210 can hold the adsorbent strip or pad. The adsorbent strip or pad can, for example, be either permanently or not permanently secured to the feminine hygiene garment 200 using adhesive, stitching, or other bonding technique. US Patent Application number 2003/0088955, incorporated in its entirety herein by reference, shows various representative feminine hygiene garment configurations that can be employed in the practice of this invention, as well as general methodologies to make such feminine hygiene garments.

### Semi-Disposable Athletic Apparel

To date, nonwovens that are used for disposable applications do not have reusable capabilities (i.e., potential for multiple wash/dry cycles) and do not have the aesthetics of normal knitted textiles.

The present inventors have recognized that the use of the above-described elastic nonwoven fabric provides a look and feel like that of a traditional knitted fabric, and be reused (washed and dried through at least 5 cycles without significant loss of properties), has functional elasticity, and is disposable, and its combination with an absorptive core could be used to produce adult incontinence garments, feminine hygiene garments, and semi-disposable garments including sweat control garments. The elastic nonwoven of this invention can be used by itself to produce a portion of a garment or to produce the entire garment.

Representative semi-disposable athletic garments of this invention include but are not limited to pants, shorts, bibs, jackets, socks, and shirts including long sleeve shirts, short sleeve shirts, and sleeve-less shirts. Semi-disposable garments intended for medical applications are also within the scope of this invention. At least a portion of these garments are made of the elastic nonwoven of this invention, and in one embodiment are entirely made of the elastic nonwoven of this invention.

### Sweat Control Garments

The sweat control garments of this invention can resemble the semi-disposable athletic garments described above. However, in a sweat control garment, at least a portion of the garment is formed of a sweat control fabric, which can be sewn in or made a panel of the garment in, for example, the back, chest, and/or arm-pit areas of a shirt.

Further modifications and alternative embodiments of this invention will be apparent to those skilled in the art in view of this description. Accordingly, this description is to be construed as illustrative only and is for the purpose of teaching those skilled in the art the manner of carrying out the invention. It is to be understood that the forms of the invention herein shown and described are to be taken as illustrative embodiments. Equivalent elements or materials may be substituted for those illustrated and described herein, and certain features of the invention may be utilized independently of the use of other features, all as would be apparent to one skilled in the art after having the benefit of this description of the invention.

1. A method for producing a garment, comprising forming at least a portion of the garment from an elastic nonwoven web, wherein the nonwoven web comprises a plurality of multicomponent strands having first and second polymer components longitudinally coextensive along the length of the strands, said first component comprising an elastomeric polymer, and said second polymer component comprising a polymer less elastic than the first polymer component.

2. The method of claim 1, wherein the nonwoven web is composed of spunbond strands.

3. The method of claim 1, wherein the first polymer component comprises an elastomeric polyurethane, elastomeric polyethylene copolymers, elastomeric polypropylene copolymers, elastomeric styrenic block polymers, or blends thereof, and the second polymer component comprises a polyolefin that is less elastic than the elastomeric polymer.

4. The method of claim 1 wherein the second polymer component is polypropylene, polyethylene, polyester, or a blend thereof.

5. The method of claim 1, wherein elastic nonwoven web is composed of fibers having a sheath/core configuration.

6. The method of claim 1, wherein the garment is in the form of an adult incontinence garment, a feminine hygiene garment, a semi-disposable athletic garment, or a sweat-control garment.

7. The method of claim 1, further comprising dyeing the elastic nonwoven web.

8. A semi-disposable article of clothing made from an elastic nonwoven web, wherein the elastic nonwoven web comprises a plurality of multicomponent strands having first and second polymer components longitudinally coextensive along the length of the strands, said first component comprising an elastomeric polymer, and said second polymer component comprising a polymer less elastic than the first polymer component.

9. The article of claim 8, wherein the article of clothing is in the form of a pair of pants.

10. The article of claim 8, wherein the article of clothing is in the form of a pair of shorts.

11. The article of claim 8, wherein the article of clothing is in the form of a shirt.

12. The article of claim 8, wherein the article of clothing is in the form of a jacket.

13. The article of claim 8, wherein the article of clothing is in the form of men's or women's underwear.

14. The article of claim 8, wherein the article of clothing is in the form of a bra.

15. The article of claim 8, wherein the article of clothing is adapted for sweat-control.

16. The article of claim 8, wherein the nonwoven web is formed by a spunbond process.

17. The article of claim 8, wherein the first polymer component comprises an elastomeric polyurethane, elastomeric polyethylene copolymers, elastomeric polypropylene copolymers, elastomeric styrenic block polymers, or blends thereof, and the second polymer component comprises a polyolefin or polyester that is less elastic than the elastomeric polymer.

18. The article of claim 8, wherein the second polymer component is polypropylene, polyethylene, polyester, or a blend thereof.

19. The article of claim 8, wherein the elastic nonwoven web is composed of fibers having a sheath/core configuration.

20. An adult incontinence garment, comprising an elastic nonwoven web in the form of a feminine hygiene product and adapted for receipt of a disposable absorbent device, wherein the elastic nonwoven web comprises a plurality of multicomponent strands having first and second polymer components longitudinally coextensive along the length of the strands, said first component comprising an elastomeric polymer, and said second polymer component comprising a polymer less elastic than the first polymer component.

21. The garment of claim 20, wherein the nonwoven web is formed by a spunbond process.

22. The garment of claim 20, wherein the first polymer component comprises an elastomeric polyurethane, elastomeric polyethylene copolymers, elastomeric polypropylene copolymers, elastomeric styrenic block polymers, or blends thereof, and the second polymer component comprises a polyolefin or polyester that is less elastic than the elastomeric polymer.

23. The garment of claim 20, wherein the second polymer component is polypropylene, polyethylene, polyester, or a blend thereof.

24. The garment of claim 20, wherein the elastic nonwoven web is composed of fibers having a sheath/core configuration.

25. A feminine hygiene product, comprising an elastic nonwoven web in the form of a feminine hygiene product and adapted for receipt of a disposable absorbent device, wherein the elastic nonwoven web comprises a plurality of multicomponent strands having first and second polymer components longitudinally coextensive along the length of the strands, said first component comprising an elastomeric polymer, and said second polymer component comprising a polymer less elastic than the first polymer component.

26. The produce of claim 25, wherein the first polymer component comprises an elastomeric polyurethane, elastomeric polyethylene copolymers, elastomeric polypropylene copolymers, elastomeric styrenic block polymers, or blends thereof, and the second polymer component comprises a polyolefin or polyester that is less elastic than the elastomeric polymer.

27. The produce of claim 25, wherein the second polymer component is polypropylene, polyethylene, polyester, or a blend thereof.

28. The produce of claim 25, wherein the elastic nonwoven web is composed of fibers having a sheath/core configuration.

29. The product of claim 25, wherein the nonwoven web is formed by a spunbond process.
